(19) United States
(12) Patent Application Publication
(10) Pub. No.: US 2008/0097348 A1
(43) Pub. Date: Apr. 24, 2008

(54) Enteral Feeding Probe and Tube System for Enteral Feeding and Gastric Decompression or Drainage

(76) Inventor: Martin Itrich, Konstanz (DE)

Correspondence Address: KENYON & KENYON LLP, ONE BROADWAY, NEW YORK, NY, 10004, US

(21) Appl. No.: 11/596,032

(22) PCT Filed: Apr. 9, 2005
(86) PCT No.: PCT/EP05/03754

§ 371 Date: Jun. 11, 2007

(30) Foreign Application Priority Data
May 11, 2004 (DE) 10 2004 023 078.1

(51) Int. Cl. A61M 23/00

(52) U.S. Cl. 604/264

(57) ABSTRACT

The invention relates to an enteral feeding tube comprising a jejunal tube which is arranged in jejunum and comprises a proximal end, a distal end and a lumen between said two ends. The jejunal tube comprises at least two layers, including an outer first layer and a second layer adjacent to the outer first layer, the outer first layer comprising a material which is harder than the material of the second layer. The jejunal tube may have a triple-layer structure that includes an outer layer, an inner layer, and an internal layer, the internal layer may be softer than the outer and inner layers. The multiple-layer structure of the jejunal tube having materials of different hardnesses improves the flexibility, advancing behavior and buckling resistance of the feeding tube. The invention also relates to a tube system including the enteral feeding tube and a gastric tube.

### FIELD OF INVENTION

The invention relates to a probe for enteral feeding of a patient with a jejunal tube for placement in the jejunum. The invention furthermore relates to a probe system with an enteral feeding probe and a gastric probe for gastric decompression or drainage.

### BACKGROUND OF INVENTION

It is known for a patient to be supplied with a nutrient solution via a probe tube that is placed in the gastrointestinal tract. With the nutrient solution, the patient can be fed in a fully balanced manner.

For decompression of the stomach, it may be necessary to place a gastric probe next to the feeding probe. Where the gastric probe is placed in conjunction with a feeding probe, it is known for the gastric probe to be placed coaxially over the feeding probe. For this, the gastric probe should have an internal diameter that is as large as possible and/or the feeding probe should have an outer diameter that is as small as possible, as the annular space between the gastric probe and the feeding probe should be as large as possible in order to be able to empty the stomach.

A probe system with a feeding probe and a gastric probe is known from DE 101 52 788 A1. First of all, the feeding probe is positioned with the aid of an endoscope. For this, the feeding probe is inserted into the working channel of the endoscope until the distal end of the jejunal tube is positioned in the jejunum. After the endoscope has been removed, the gastric probe is pushed onto the feeding probe until the distal end of the gastric probe is positioned in the gastrointestinal tract.

In order to ensure secure advancement and secure positioning of the feeding probe, it is known for a guide wire to be provided for the feeding probe. This guide wire is intended to lend the feeding probe the necessary rigidity. A so-called “zebra wire” of a NiTi alloy with a PTFE shell is known as a guide wire. The endoscopic placement of the feeding probe in conjunction with a gastric probe whilst using a guide wire is however problematic in that moving the instruments and tubes relative to one another must not lead to any positional change (dislocation) of the feeding probe in the gastrointestinal tract.

Further probes are known from EP 1 266 646 A2 and EP 0 354 695 A2. EP 1 266 646 A2 describes a probe system with a feeding probe and a gastric probe. The gastric probe has a two-layer structure with an outer layer of a soft, flexible plastic and an inner layer of a plastic that is harder in relation to the plastic of the outer layer. The outer layer of the gastric probe, which in itself is unstable, is intended to be stabilized by the inner layer of harder plastic. Furthermore, EP 1 266 646 A2 proposes that the surface of the feeding probe be configured with a microroughness to improve the sliding properties. A catheter with a harder inner layer and a softer outer layer is known from EP 0 354 695 A2.

### SUMMARY OF THE INVENTION

An object of the invention is to provide a feeding probe which can be positioned without the use of a guide wire, in particular in conjunction with a gastric probe. A further object of the invention is to create a probe system with a feeding tube and a gastric tube which can be positioned without the use of a guide wire.

In an embodiment of the invention an enteral feeding probe has a probe tube with a structure of at least two layers, an outer first layer and a second layer that is adjacent to the outer layer. The material of the first layer is harder than the material of the second layer. It has been shown that the harder outer layer ensures sufficient stability to allow positioning of the probe without further aids. At the same time, the outer harder layer reduces sliding friction, so that the gastric probe can be pushed over the feeding probe more easily. Additionally, the inner layer that is adjacent to the outer layer ensures the necessary flexibility. The combination of the two layers of different hardness, on the one hand one achieves improved flexibility and on the other hand one achieves improved advancing behavior along with improved resistance to buckling.

In an embodiment of the invention, the probe tube has a structure of three layers including an outer first layer, an enclosed second layer that is adjacent to the outer layer, and an inner third layer that is adjacent to the enclosed layer. The inner third layer comprises a material that is harder than the material of the enclosed layer. The materials of the inner layer and the outer layer may be of the same hardness. It has been shown that this embodiment has further improved sliding and advancing properties.

The material of the outer layer may have a Shore hardness according to ASTM D 2240 that is greater than 40 and less than 70 D, preferably greater than 50 and less than 60 D, in particular 55 D, whereas the material of the enclosed layer may have a Shore hardness that is greater than 70 and less than 100 D, preferably greater than 80 and less than 90 D, in particular 85 D.

In an embodiment of the invention, the surface of the outer layer is configured with a microroughness. The friction can be further reduced with the matt-finished or slightly rough surface.

Due to the improved stability, the thickness of the jejunal probe tube can be reduced. This results in a smaller outer diameter, so that the probe tube fits into the working channel of standard endoscope.

An embodiment of the probe system according to the invention includes, besides the feeding probe, a gastric probe for gastric decompression or drainage. The gastric probe has a gastric probe tube that is of a smaller length and greater diameter than the jejunal tube. Through this, it is possible to position the gastric probe tube in the gastrointestinal tract and the jejunal probe tube in the jejunum, and to insert the jejunal probe tube into the gastric probe tube. The jejunal probe tube may be more than twice the length of the gastric probe tube.

In an embodiment of the probe system according to the invention, the gastric probe tube has a structure of at least two layers. The inner layer of the gastric probe tube may include a material that is harder than the material of the outer layer. Since the outer layer is softer than the inner layer, the probe tube has on the one hand adequate stability and on the other hand sufficient suppleness in order to avoid injury to the mucous membrane when the gastric probe is inserted. In the case of a particularly preferred embodiment, the sliding properties of the gastric probe are further improved by a surface configured with microroughness.

### BRIEF DESCRIPTION OF THE DRAWINGS

FIG. 1 shows the enteral feeding probe according to the invention, in a partially sectional representation.

FIG. 2 shows a section through the feeding probe of FIG. 1, along line A-A.

FIG. 3 shows the gastric probe according to the invention, in a partially sectional representation.

FIG. 4 shows a section through the gastric probe of FIG. 3, along line B-B.

FIG. 5 shows the probe system according to the invention, with the feeding probe and gastric probe, in a partially cut-away representation.

### DETAILED DESCRIPTION

In the following, embodiment examples of the apparatus in accordance with the invention are explained in more detail by reference to the drawings.

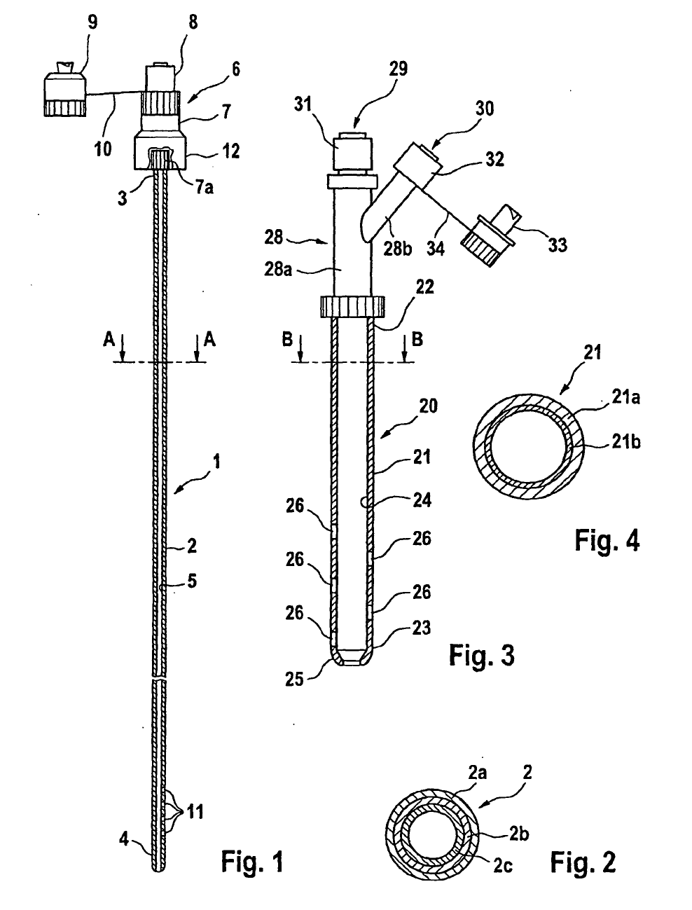

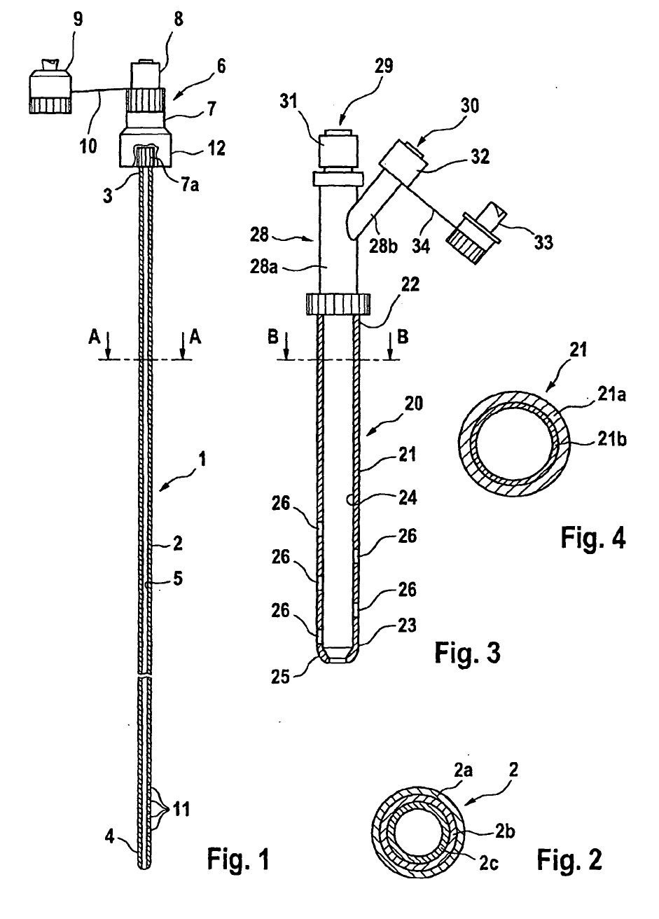

FIGS. 1 and 2 show the feeding probe and FIGS. 3 and 4 show the gastric probe.

FIG. 1 shows feeding probe 1. The feeding probe 1 has a probe tube 2 of a length of, for example, 2700 mm. Probe tube 2 has an open proximal end 3 and an open distal end 4. Between the proximal and distal ends 3, 4 runs lumen 5 of the probe tube. The probe tube has an outer diameter, for example, of approximately 2.6 mm and an inner diameter (lumen) of, for example, 1.8 mm.

Connected to the proximal end 3 of the probe tube 1 is a connection piece 6 for connecting a transfusion system (not shown) for supplying a nutrient solution. The tube conduits of the familiar transfusion systems have a complementary connection piece that is connected to the connection piece 6 of the feeding probe.

The connection piece 6 of the feeding probe 1 has a base body 7 with a Luer lock connector 8. For sealing the Luer lock connector 8, the connection piece 6 has a sealing cap 9, which seals the Luer lock connector 8 tightly. The sealing cap 9 is connected to the base body 7 of the connection piece 6 via a flexible web 10.

The connection piece 6 is held in a clamped manner on the probe tube 2 of the feeding probe such that the connection piece can be removed. For this, the base body 7 of the connection piece has a clamping piece 7a that is placed on the proximal end of the probe tube.

In the area of the distal end 4, the probe tube 2 has four lateral outlet openings 11, to allow the nutrient solution to flow out. To avoid injuries, the distal end 4 of the tube conduit is rounded off.

FIG. 2 shows a section through the feeding probe 1. The probe tube 2 has a three-layer structure. It includes an outer layer 2a of a harder material, an enclosed layer 2b of a softer material, and an inner layer 2c of a harder material. The surface of the outer layer 2a is configured with a microroughness.

The material of the outer layer 2a has a Shore hardness according to ASTM D 2240 that is greater than 50 and less than 60 D, in particular 55 D. The inner layer 2c includes a material with the same Shore hardness as the outer layer 2a. The enclosed layer 2b includes a material with a Shore hardness according to ASTM D 2240 that is greater than 80 and less than 90 D, in particular 85 D. The inner and outer layers 2a, 2c have a layer thickness of 0.1 mm, for example.

For monitoring the positioning of the feeding probe, at least one of the three layers can provide X-ray contrast. The probe tube 2 can for example comprise elastic polymers, such as thermoplastic elastomers, polyurethane, modified polyamides or polyolefins, styrene polymers, fluoropolymers, PVC with plasticizers, etc. The materials provide for the necessary Shore hardness as well as biocompatibility.

The triple-layer structure of the feeding probe according to the invention provides improved advancing behavior and greater resistance to buckling together with improved flexibility. Since the feeding tube does not use a guide wire, fewer work steps are involved when positioning the feeding probe in conjunction with the gastric probe. Also, the manufacturing costs are lower without the expensive guide wire. Furthermore, the risk of dislocation of the feeding probe is further reduced. With the triple-layer structure, the outer diameter of the jejunal probe tube can be selected such that it fits through the working channel of a standard endoscope and still allows an adequate internal diameter for the application of a nutrient solution into the small intestine. The outer diameter should preferably be smaller than 2.8 mm.

FIG. 3 shows gastric probe 20. The gastric probe 20 has a gastric probe tube 21 of a length of 1200 mm with an open proximal end 22 and an open distal end 23. Lumen 24 of the probe tube 21 extends between the proximal and distal ends 22, 23. The distal end 23 of the probe tube is closed off with an olive 25 which is open at the end, and which can provide X-ray contrast. Lateral openings 26 are arranged distributed around the circumference in the area of the distal end 23 of the probe tube 21.

The inner diameter of the probe tube 21 of the gastric probe 20 is greater than the outer diameter of the probe tube 2 of the feeding probe 1, so that the probe tube of the gastric probe 20 can be pushed onto the probe tube of the feeding tube 1 leaving an annular gap with an adequate cross section. In the present example, the outer diameter of the gastric probe tube 21 is 5.2 mm and the inner diameter is 3.9 mm.

Connected to the proximal end 22 of the gastric probe tube 21 is a connection piece 28. The connection piece 28 has a first access point 29 for receiving the jejunal probe tube 2 and a second access point 30 for connecting a suction line (not shown). The connection piece 28 of the gastric probe 20 is preferably a Y-shaped adapter with a straight connection piece 28a and a connection piece 28b that branches off from the straight connection piece. Connected to the straight connection piece 28a is a first connector 31 for connecting the connection piece 6 of the feeding probe 1, and connected to the branching connection piece 28b is a second connector 32 for connecting the suction line. The connector 32 for connecting the suction line is for example a Luer lock connector. The Luer lock connector 32 can be tightly sealed with a suitable sealing cap 33, which is joined to the branching connection part 28b via a flexible web 34.

The connection piece 6 of the feeding probe 1 and the connection piece 28 of the gastric probe 20 can be placed to fit on one another when the gastric probe is pushed onto the feeding probe. For this, the connection piece 6 of the feeding probe 1 has a cylindrical base part 12 which can be placed to fit onto the connector 31 of the connection piece 28 of the gastric probe 20. The base part 12 and the connector 31 are designed in such a way that the connection piece 6 of the feeding probe 1 and the connector 31 of the connection piece 28 of the gastric probe 20 form a snap connection. For this, there can for example be grooves or channels provided on the exterior of the connector 31 and the interior of the base part 12, which engage to fit with one another so that a secure but easily released connection is created.

FIG. 4 shows a section through the probe tube 20. The probe tube 21 of the gastric probe 20 has a two-layer structure. The gastric probe tube 21 can be of the same material as the jejunal probe tube 2. The outer layer 21a of the gastric probe tube 21 comprises a material with a Shore hardness according to ASTM D 2240 that is preferably greater than 70 and less than 90 D, in particular 85 D. The inner layer comprises a material with a Shore hardness preferably 50 to 70 D, in particular 55 D. The outer layer 21a has a greater thickness than the inner layer 21b, with the inner layer for example having a thickness of 0.1 to 0.2 mm. Since the outer layer 21a is softer than the inner layer 21b, the gastric probe has a less traumatic effect on the oesophagus, with sufficient stability. The inner surface of the inner layer 21b can once again be configured with a microroughness.

FIG. 5 shows the feeding probe 1 in conjunction with the gastric probe 20. First, the feeding probe 1 is positioned with the aid of an endoscope. Here, the connection piece 6 is not placed on the probe tube 2. Then the endoscope is withdrawn, which is not possible with the connection piece in place. After that, the probe tube 21 of the gastric probe 20 is pushed over the probe tube 2 of the feeding probe 1. The probe tube 2 of the feeding probe 1 is subsequently shortened at the proximal end to the correct length, and the connection piece 6 is placed on the proximal end of the probe tube 2, or the first connector 31 of the gastric probe 20. The transfusion system (not shown) for supplying the nutrient solution and the suction line (not shown) for decompression of the stomach can then be connected.

1-20. (canceled)

21. An apparatus for enteral feeding, comprising: a jejunal probe tube including a proximal end, a distal end and a lumen between the proximal end and the distal end, wherein the jejunal probe tube has a structure of at least two layers including an outer first layer and a second layer that is adjacent to the outer first layer, wherein the outer first layer comprises a material that is harder than the material of the second layer.

22. The apparatus of claim 21, wherein the jejunal probe tube has a structure of three layers, including an outer first layer, an enclosed second layer that is adjacent to the outer layer, and an inner third layer that is adjacent to the enclosed second layer, wherein the third layer comprises a material that is harder than the material of the second layer.

23. The apparatus of claim 22, wherein the materials of the outer first layer and the inner third layer of the jejunal probe tube are of the same hardness.

24. The apparatus of claim 21, wherein the material of the outer first layer of the jejunal probe tube has a Shore hardness according to ASTM D 2240 that is greater than 40 and less than 70 D.

25. The apparatus of claim 21, wherein the material of the outer first layer of the jejunal probe tube has a Shore hardness according to ASTM D 2240 that is greater than 50 and less than 60 D.

26. The apparatus of claim 21, wherein the material of the outer first layer of the jejunal probe tube has a Shore hardness according to ASTM D 2240 that is approximately 55 D.

27. The apparatus of claim 22, wherein the material of the outer first layer of the jejunal probe tube has a Shore hardness according to ASTM D 2240 that is greater than 40 and less than 70 D.

28. The apparatus of claim 23, wherein the material of the outer first layer of the jejunal probe tube has a Shore hardness according to ASTM D 2240 that is greater than 40 and less than 70 D.

29. The apparatus of claim 21, wherein the material of the second layer of the jejunal probe tube has a Shore hardness according to ASTM D 2240 that is greater than 70 and less than 100 D.

30. The apparatus of claim 21, wherein the material of the second layer of the jejunal probe tube has a Shore hardness according to ASTM D 2240 that is greater than 80 and less than 90 D.

31. The apparatus of claim 21, wherein the material of the second layer of the jejunal probe tube has a Shore hardness according to ASTM D 2240 that is approximately 85 D.

32. The apparatus of claim 22, wherein the material of the enclosed second layer of the jejunal probe tube has a Shore hardness according to ASTM D 2240 that is greater than 70 and less than 100 D.

33. The apparatus of claim 21, wherein the outer surface of the outer first layer of the jejunal probe tube is structured with a microroughness.

34. The apparatus of claim 22, wherein the outer surface of the outer first layer of the jejunal probe tube has a microroughness.

35. The apparatus of claim 21, further comprising a first connection piece connected to the proximal end of the jejunal probe tube for connecting a tube conduit of a transfusion system for supplying a nutrient solution.

36. The apparatus of claim 21, further comprising outlet openings formed in the area of the distal end of the jejunal probe tube.

37. The apparatus of claim 21, wherein the distal end of the jejunal probe tube is rounded off.

38. The apparatus of claim 21, further comprising a gastric probe for gastric decompression or drainage, the gastric probe structured to engage the jejunal probe tube.

39. The apparatus of claim 38, wherein the gastric probe includes a gastric probe tube having a proximal end, a distal end and a lumen between the proximal end and the distal end, the jejunal probe tube being longer than the gastric probe tube and the jejunal probe tube having a smaller outer diameter than the inner diameter of the gastric probe tube, such that the jejunal probe tube can be inserted into the gastric probe tube.

40. The apparatus of claim 39, wherein the gastric probe tube of the gastric probe has a structure of at least two layers, including an outer layer and an inner layer, wherein the inner layer comprises a material that is harder than the material of the outer layer.

41. The apparatus of claim 40, wherein the material of the outer layer of the gastric probe tube has a Shore hardness according to ASTM D 2240 that is greater than 70 and less than 100 D.

42. The apparatus of claim 40, wherein the material of the outer layer of the gastric probe tube has a Shore hardness according to ASTM D 2240 that is greater than 80 and less than 90 D.

43. The apparatus of claim 40, wherein the material of the outer layer of the gastric probe tube has a Shore hardness according to ASTM D 2240 that is approximately 85 D.

44. The apparatus of claim 40, wherein the material of the inner layer of the gastric probe tube has a Shore hardness according to ASTM D 2240 that is greater than 40 and less than 70 D.

45. The apparatus of claim 40, wherein the material of the inner layer of the gastric probe tube has a Shore hardness according to ASTM D 2240 that is greater than 50 and less than 60 D.

46. The apparatus of claim 40, wherein the material of the inner layer of the gastric probe tube has a Shore hardness according to ASTM D 2240 that is approximately 55 D.

47. The apparatus of claim 40, wherein the inner surface of the inner layer of the gastric probe tube is structured with a microroughness.

48. The apparatus of claim 40, further comprising openings formed in the area of the distal end of the gastric probe tube.

49. The apparatus of claim 40, wherein the distal end of the gastric probe tube is closed off with an olive that is open at the end.

50. The apparatus of claim 40, further comprising a second connecting piece, the proximal end of the gastric probe tube connected to the second connection piece, the second connection piece including a first access point for receiving the jejunal probe tube and a second access point for connecting a suction line.

51. The apparatus of claim 50, wherein the first access point of the second connection piece of the gastric probe tube and the first connection piece of the jejunal probe tube are configured such that the first connection piece can be placed to fit on the second connection piece.

52. The apparatus of claim 51, wherein the first connection piece and the second connection piece are joinable by snapping together.
